(19) United States
(12) Patent Application Publication
(10) Pub. No.: US 2025/0380995 A1
(43) Pub. Date: Dec. 18, 2025

(54) ASSISTED PORT PLACEMENT FOR MINIMALLY INVASIVE OR ROBOTIC ASSISTED SURGERY

(71) Applicant: Covidien LP, Mansfield, MA (US)

(72) Inventors: Meir Rosenberg, Newton, MA (US); Faisal I. Bashir, Andover, MA (US); Rainer C. Konietschke, Wessling (DE); Max L. Balter, Boston, MA (US); Tuvia C. Rappaport, Somerville, MA (US); Michael A. Eiden, Somerville, MA (US)

(21) Appl. No.: 18/875,840

(22) PCT Filed: Jun. 27, 2023
(86) PCT No.: PCT/US2023/069127

§ 371 Date: Dec. 17, 2024

Related U.S. Application Data
(60) Provisional application No. 63/355,713, filed on Jun. 27, 2022.
(60) Provisional application No. 63/440,985, filed on Jan. 25, 2023.

(51) Int. Cl. A61B 34/30; A61B 1/00; A61B 1/313; A61B 17/34; A61B 34/00; A61B 34/10; A61B 34/20

(52) U.S. Cl. CPC A61B 34/30; A61B 1/00009; A61B 1/00048; A61B 1/0005; A61B 1/00149; A61B 1/3132; A61B 17/3423; A61B 2034/105; A61B 2034/107; A61B 2034/2055; A61B 2034/2059; A61B 2034/252; A61B 2034/302

(57) ABSTRACT

A surgical robotic system includes a robotic arm holding a laparoscopic camera inserted through an access port. The system also includes a controller configured to generate a port location for an access port on a 3D model of a patient and generate a patient-specific setup guide for configuring the access port and the robotic arm. The system also includes an external camera configured to register the robotic arm and the patient. The system further includes a display configured to output the port location of the access port as an overlay over an external image of the patient based on registration of the robotic arm and the patient.

### CROSS REFERENCE TO RELATED APPLICATIONS

This application claims the benefit of and priority to U.S. Provisional Patent Application Ser. No. 63/355,713 filed Jun. 27, 2022, and U.S. Provisional Patent Application Ser. No. 63/440,985, filed Jan. 25, 2023. The entire disclosures of the foregoing applications are incorporated by reference herein.

### BACKGROUND

Minimally invasive surgery (MIS) and robotic assisted surgery (RAS) use single or multiple incisions on external human anatomy to access the surgical site. The locations of these incisions determine where access ports (or trocars) are placed. Surgeons and assistants insert instruments, including the laparoscopic camera, through these ports to access the surgical site. The current practice is for surgeons to palpate and visually locate the anatomical landmarks for patient placed on bed, e.g., rib cage and umbilicus. A fixed port placement guidance template is used to make incisions and place ports on the patient's external anatomy. Advanced users skilled in the art of surgery can perform this procedure efficiently for either MIS or for the RAS platform for which they have been pre-trained. There is an unmet need for assisted port placement for novice users of existing MIS or RAS platforms, and all users of the new RAS platforms. Setup time for robotic surgical systems may be lengthy and cumbersome and depends on placement of access ports. Thus, there is a need for systems for virtual placement of access ports, which is then used to determine initial placement of movable carts and robotic arms.

### SUMMARY

According to one embodiment of the present disclosure, a surgical robotic system is disclosed The surgical robotic system includes a robotic arm holding a laparoscopic camera inserted through an access port. The system also includes a controller configured to generate a port location for an access port on a 3D model of a patient and generate a patient-specific setup guide for configuring the access port and the robotic arm. The system also includes an external camera configured to register the robotic arm and the patient. The system further includes a display configured to output the port location of the access port as an overlay over an external image of the patient based on registration of the robotic arm and the patient.

Implementations of the above embodiment may include one or more of the following features. According to one aspect of the above embodiment, the display may be a monitor or a head-mounted display. The external camera may be further configured to capture a plurality of external images of a patient and images of the robotic arm. The laparoscopic camera may be configured to capture internal images of a surgical site. The controller may be further configured to generate a depth map of the surgical site from the internal images of the surgical site. The controller may be further configured to generate a registration of preoperative imaging data of the patient with the depth map. The controller may be additionally configured to track location of the laparoscopic camera via kinematics of the robotic arm and visual-simultaneous localization and mapping (visual-SLAM). The controller may be further configured to update the registration between the preoperative imaging data with the depth map via fully automatic registration using real-time robotic arm kinematics data and the visual-SLAM.

According to another embodiment of the present disclosure, a method for assisted access port placement is disclosed. The method includes inserting a laparoscopic camera held by a robotic arm through an access port. The method also includes generating, at a controller, a port location for an access port on a 3D model of a patient and generating a patient-specific setup guide for configuring the access port and the robotic arm. The method further includes registering the robotic arm and the patient at an external camera and outputting on display the port location of the access port as an overlay over an external image of the patient based on registration of the robotic arm and the patient.

Implementations of the above embodiment may include one or more of the following features. According to one aspect of the above embodiment, the port location of the access port may be output as the overlay on at least one of a monitor or a head-mounted display. The method may also include capturing a plurality of external images of a patient and images of the robotic arm through the external camera. The method may additionally include capturing internal images of a surgical site through the laparoscopic camera. The method may also include generating, at the controller, a depth map of the surgical site from the internal images of the surgical site. The method may further include generating, at the controller, a registration of preoperative imaging data of the patient with the depth map. In aspects, the method may further include tracking location of the laparoscopic camera via kinematics of the robotic arm and visual-simultaneous localization and mapping (visual-SLAM). The method may further include tracking the registration between the preoperative imaging data with the depth map via fully automatic registration using real-time robotic arm kinematics data and the visual-SLAM.

According to a further embodiment of the present disclosure, a method for determining access port placement is disclosed. The method includes capturing a plurality of external images of a patient from an external vision system and generating an external 3D model of a patient based on the plurality of external images. The method also includes generating an internal 3D model of the patient based on preoperative imaging data and generating a combined 3D model based on the external 3D model and the internal 3D model. The method further includes determining a port location for at least one access port based on the combined 3d model and generating a setup guide for configuring at least one access port and a robotic arm. The method additionally includes outputting on a display the port location of the at least one access port and the setup guide as an overlay over an external image of the patient based on registration of the robotic arm and the patient.

Implementations of the above embodiment may include one or more of the following features. According to one aspect of the above embodiment, the method may also include generating the external 3D model of the patient based on a depth map of the patient. The method may further include generating a skeleton model may include a plurality of keypoints for the patient based on the plurality of external images. The method may additionally include generating the external 3D model based on the skeleton model.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an embodiment of the present disclosure;

FIG. 6 is a flowchart of a method for generating a setup guide for assisted port placement according to an embodiment of the present disclosure;

FIG. 7 is a computed tomography image according to an embodiment of the present disclosure;

FIG. 8 shows a white light (i.e., RGB) still image and a counterpart depth map image of the surgical robotic system of FIG. 1 and a patient according to an embodiment of the present disclosure;

FIG. 9 is a schematic flow chart of pose estimation of the patient according to an embodiment of the present disclosure;

FIG. 10 is a computer-generated 3D model of a patient including a skeleton model according to an embodiment of the present disclosure;

FIG. 11 is an image of a graphical user interface (GUI) including the 3D model of a patient and suggested access port locations according to an embodiment of the present disclosure;

FIG. 12 is the image of the GUI including port locations for the instrument and the camera according to an embodiment of the present disclosure;

FIG. 13 is the image of the GUI including preview windows of the camera at different orientations according to an embodiment of the present disclosure;

FIG. 14 is a flow chart of a method for assisted port placement based on the setup guide according to an embodiment of the present disclosure;

FIG. 15 is a perspective view of a virtual reality system for assisted port placement according to an embodiment of the present disclosure;

FIG. 16 is an image of projection of internal organs and port locations according to an embodiment of the present disclosure; and

FIG. 17 is an image of projection of internal organs and an access port according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives user input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands to control one or more actuators of the robotic arm, which would, in turn, move the robotic arm and the instrument in response to the movement commands.

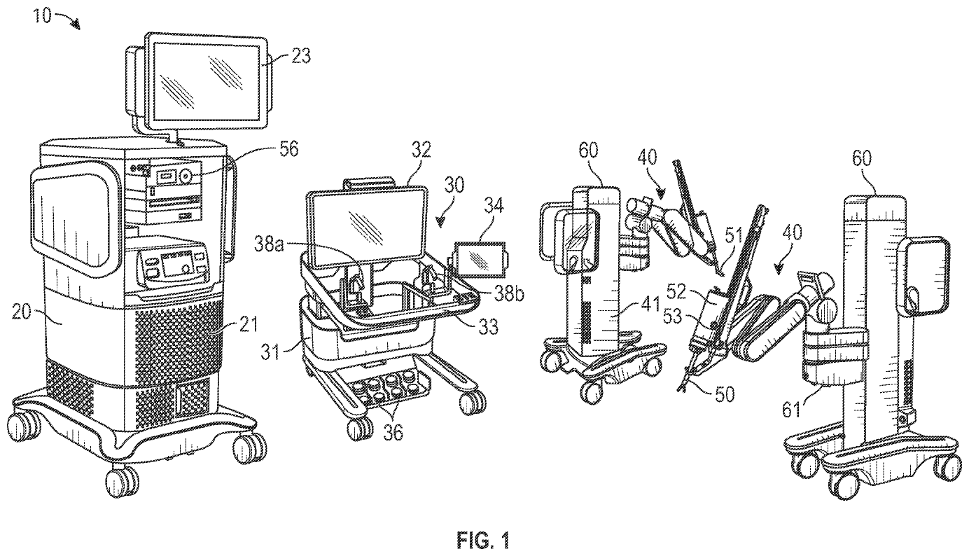

With reference to FIG. 1, a surgical robotic system 10 includes a control tower 20, which is connected to all the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include a laparoscopic camera 51 configured to capture video of the surgical site. The laparoscopic camera 51 may be a stereoscopic endoscope configured to capture two side-by-side (i.e., left and right) images of the surgical site to produce a video stream of the surgical scene. The laparoscopic camera 51 is coupled to a video processing device 56, which may be disposed within the control tower 20. The video processing device 56 may be any computing device as described below configured to receive the video feed from the laparoscopic camera 51 and output the processed video stream.

The surgeon console 30 includes a first display 32, which displays a video feed of the surgical site provided by camera 51 disposed on the robotic arm 40, and a second display 34, which displays a user interface for controlling the surgical robotic system 10. The first display 32 and second display 34 may be touchscreens allowing for displaying various graphical user inputs.

The surgeon console 30 also includes a plurality of user interface devices, such as foot pedals 36 and a pair of handle controllers 38a and 38b which are used by a user to remotely control robotic arms 40. The surgeon console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding instrument 50 or camera 51 attached thereto. This allows the user to reposition the hand controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a graphic processing unit (GPU) a microprocessor, and combinations thereof. Those skilled in the art will appreciate that the processor may be substituted for by using any logic processor (e.g., control circuit) adapted to execute algorithms, calculations, and/or set of instructions described herein.

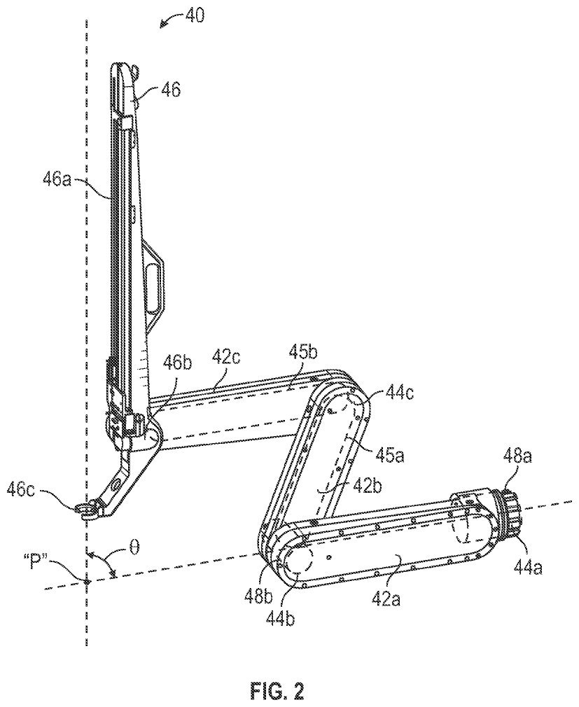

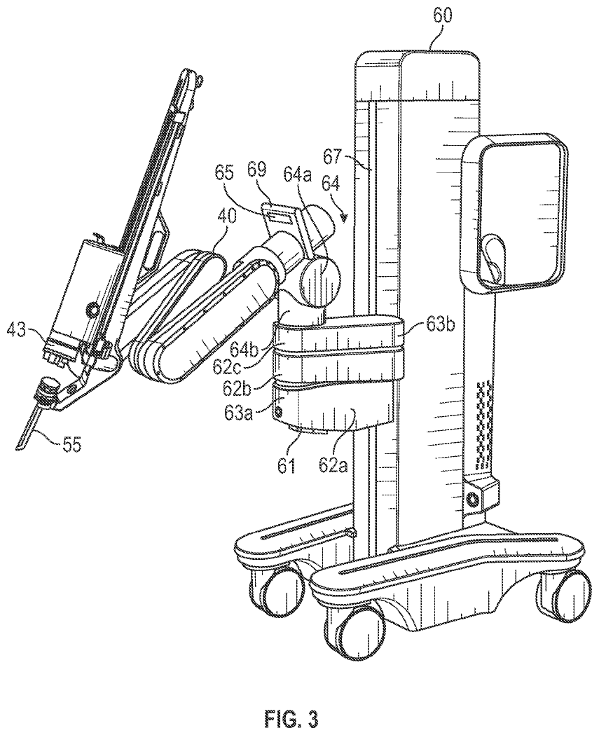

With reference to FIG. 2, each of the robotic arms 40 may include a plurality of links 42a, 42b, 42c, which are interconnected at joints 44a, 44b, 44c. respectively. Other configurations of links and joints may be utilized as known by those skilled in the art. The joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle 0 between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted to achieve the desired angle 0. In embodiments, some, or all the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components an end effector of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

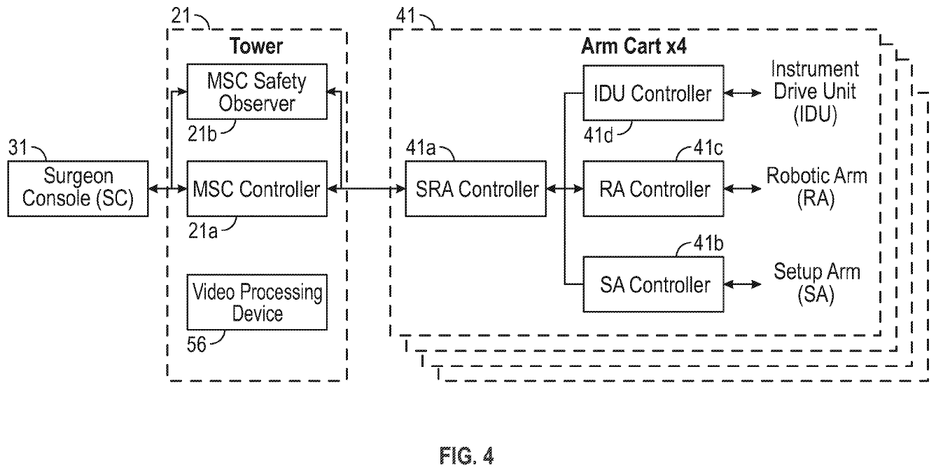

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

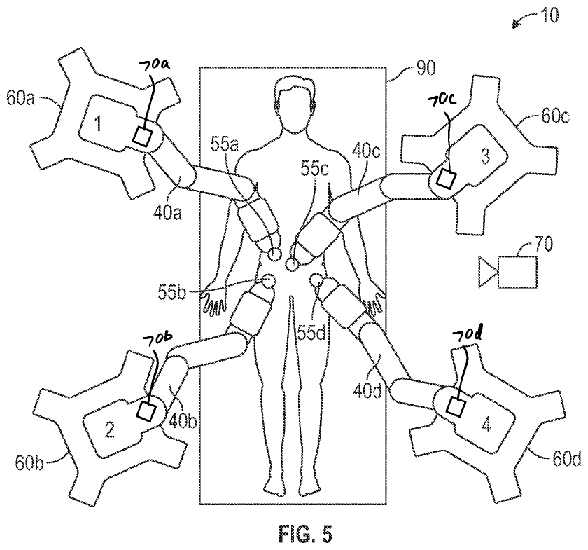

With reference to FIG. 5, the surgical robotic system 10 is setup around a surgical table 90. The system 10 includes movable carts 60a-d, which may be numbered “1” through “4.” During setup, each of the carts 60a-d is positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the laparoscopic camera 51 into corresponding ports 55a-d. During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then calibrated by the robotic arm 40 and is inserted through the access port 55 by moving the IDU 52 along the holder 46.

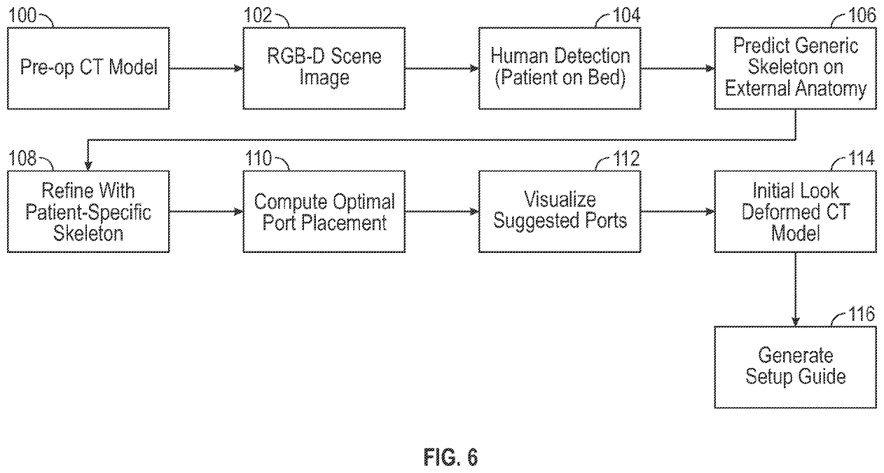

With reference to FIG. 6, a method for assisted port placement may be implemented as software instructions executable by a processor (e.g., controller 21a) and as a software application having a graphical user interface (GUI) for planning a surgical procedure to be performed by the robotic system 10. The software receives as input various 3D image and position data pertaining to the patient's anatomy and generates a 3D virtual model of the patient with suggested placements for the camera 51 and the instruments 50. The software application also allows a user to manipulate computer models of the camera 51 and the instrument 50 in the model of the patient to view the endoscopic view of the camera 51.

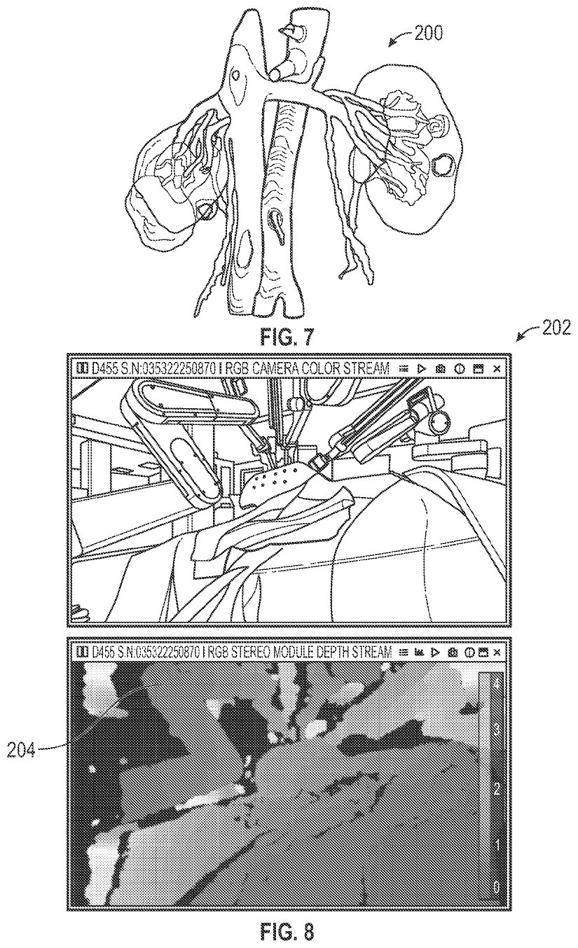

At step 100, preoperative internal imaging is obtained of the patient, which may be performed using any suitable imaging modality such as computed tomography (CT), magnetic resonance imaging (MRI), or any other imaging modality capable of obtaining 3D images. The preoperative images are then be used to construct an internal tissue and organ model 200 as shown in FIG. 7. The internal 3D model 200 may be constructed using any suitable image processing computer.

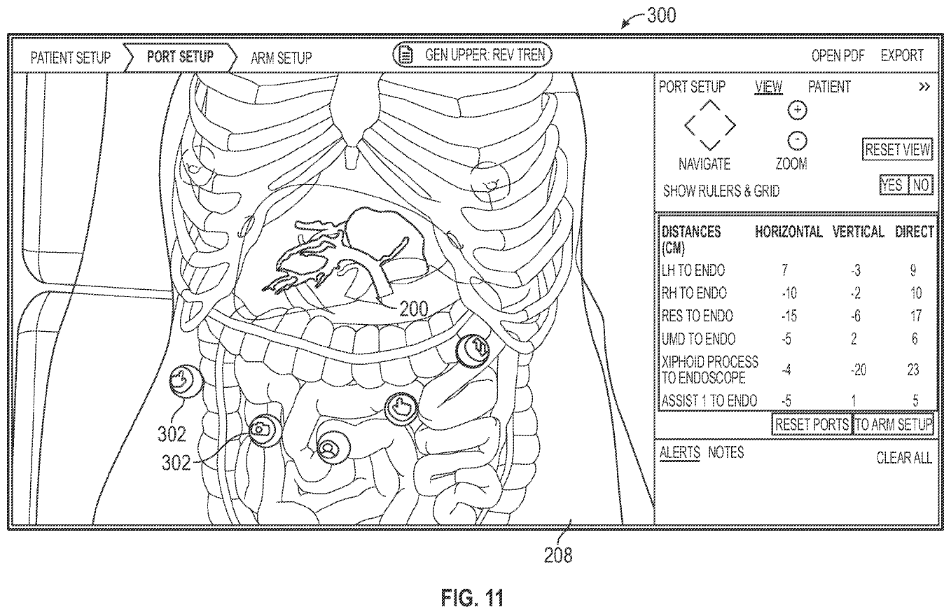

At step 102, an external image 202 is obtained of the patient's body and a depth map 204 is generated from the image 202 as shown in FIG. 8. The image 202 may be obtained using an external vision system 70 (FIG. 5), which may be a passive stereoscopic camera to provide for depth imaging as well to generate the depth map 204. In embodiments, the external vision system 70 may be an active stereoscopic infrared (IR) camera having an IR module configured to project an array of invisible IR dots onto the patient. The external vision system 70 is configured to detect the dots and analyze the pattern to create the depth map 204, which may then be used to create an external 3D model 208 of the patient. In further embodiments, the external vision system 70 may be a time-of-flight camera having an IR laser module that paints the scene with short bursts of IR lasers and generates dense depth map based on the time it takes for each laser emission to be received back at the detector. In further embodiments, the external vision system 70 may be the multi-camera subsystem of an augmented reality headset worn by the clinical staff. The external vision system 70 may also be used as a user input system to monitor user hand gestures that are then processed to determine desired user input, e.g., to manipulate a GUI 300 of FIGS. 11-13.

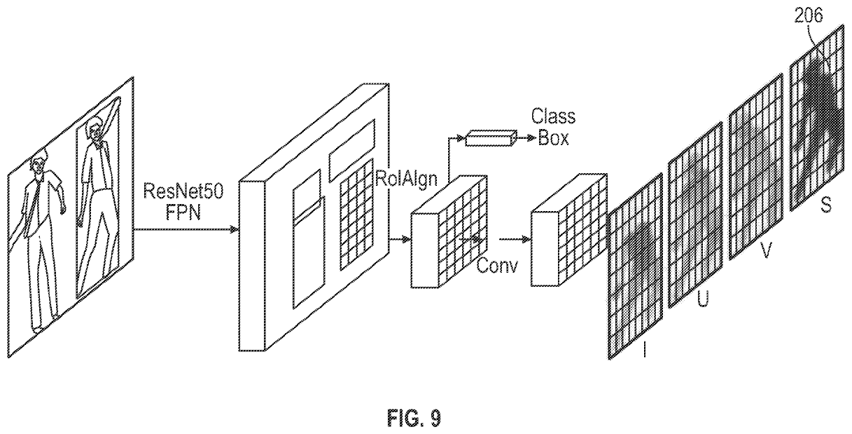

At step 104, a pose 206 of the patient is estimated by the controller 21a using machine learning image processing algorithms as shown in FIG. 9. In embodiments, machine learning may include a convolutional neural network (CNN) and/or a vision transformer (ViT) backbone to extract features along with a lightweight decoder for pose estimation. The CNN or Vi'T may be trained on previous data, for example, synthetic and/or real images of patients in various poses.

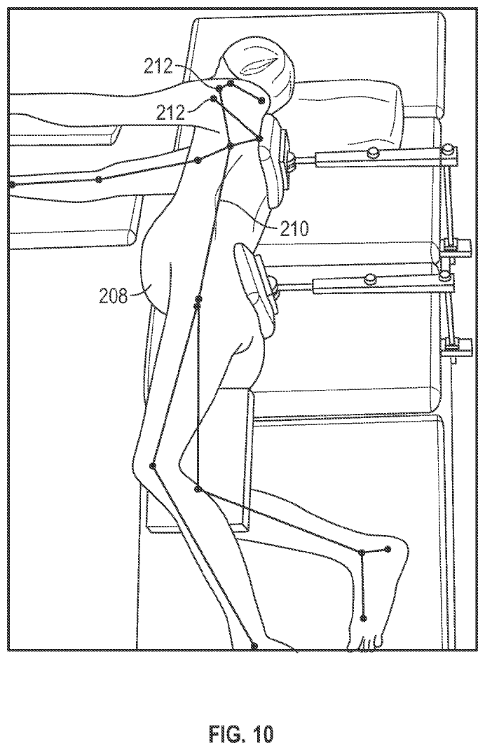

At step 106, the controller 21a predicts a skeleton for the patient. The prediction may be used to generate an external 3D model 208 of the patient as shown in FIG. 10. The external 3D model 208 includes a virtual skeleton model 210 with a plurality of keypoints 212 corresponding to the joints of the patient's anatomy. The external 3D model 208, the skeleton model 210, and the keypoints 212 are based on the external image 202, the depth map 204, and the pose 206. The keypoints 212 may be generated using various machine learning algorithms trained on datasets including synthetic and/or real images of patients in various poses.

At step 108, the external 3D model 208 is further refined to the patient specific skeleton. The patient-specific keypoint refinement algorithm may rely on the pre-operative imaging (CT/MRI) scans. The pre-operative imaging data may be segmented at different levels of densities in order: A first level of processing may generate the low-density soft-tissue external anatomical regions of the patient from the pre-operative imaging data; A second level of processing may generate the high-density internal bony joints of the patient from the pre-operative imaging data. The pre-operative imaging data (CT/MRI) is thus used to adjust the position of the keypoints 212. The 3D model may then be fitted around the refined skeleton model 210. The external 3D model 208 may also be based on patient body habitus input into the software application. The controller 21a may receive a user input for modifying the simulated patient habitus and modify the simulated patient habitus based on the user input. The user input may include sliders or other inputs to adjust patient habitus dimensions, body positions, and/or leg/arm positions. Furthermore, the initial state of the sliders or other inputs may be automatically adjusted based on the refined skeleton model 210.

At step 110, the controller 21a is configured to determine optimal access port (i.e., access port 55) locations 302 based on the procedure being performed, e.g., organ being operated on. For example, partial nephrectomy involves different port placement than radical prostatectomy, etc. Port locations 302 are also determined based on the specific anatomy of the patient, which in turn, is based on the patient's internal 3D model 200 and external 3D model 208. The port locations are determined by an optimization algorithm based on the initial fixed port placement guides for the specific procedure, the patient-specific external and internal models, the suggested instruments and their kinematics, and the robotic arms model. The optimization algorithm may start with the initial static port placement guides as starting point. The optimization algorithm may include a reinforcement learning algorithm in which the environment is in the form of the patient anatomy to be operated on, the algorithm generates a set of port locations as actions, and the reward is measured in the form of collision-free optimal access to patient anatomy. The optimization algorithm may present the user with multiple equally optimal port location plans letting the user select one.

At step 112, and as shown in FIGS. 11-13, the optimal port locations 302 are shown in a GUI 300 of the software application. The GUI 300 displays the external 3D model 208 combined with the internal 3D model 200 including the port locations 302. The external 3D model 208 may be partially transparent to display the internal 3D model 200 including patient's organs, which may be color coded for easy identification. The GUI 300 is used to generate, view, and modify the port locations 302 based on the patient's internal 3D model 200 and external 3D model 208. Furthermore, the GUI includes the 3D geodesic distance between different ports computed on the patient anatomy in real-time. The distance between ports is displayed to the clinical staff to ensure that ports are placed at least a minimum distance away from each other to minimize the likelihood of arm collisions. Additionally, the GUI may display distance between each of the port locations to the organs of interest based on the internal 3D model 200.

The GUI 300 allows for movement of the port locations 302 along the outside surface of the external 3D model 208. The user may select the port locations 302 in need of modification by simulating operation of instruments 50 and/or camera 51 being inserted through the access ports 55a-d at the port locations 302. Furthermore, if the clinical staff moves the ports such that the distance between any two ports is less than threshold distance, the GUI may display a warning. Additionally, the GUI may display virtual boundaries around the suggested port locations such that moving the ports within the virtual boundaries would still satisfy all the optimal port placement constraints.

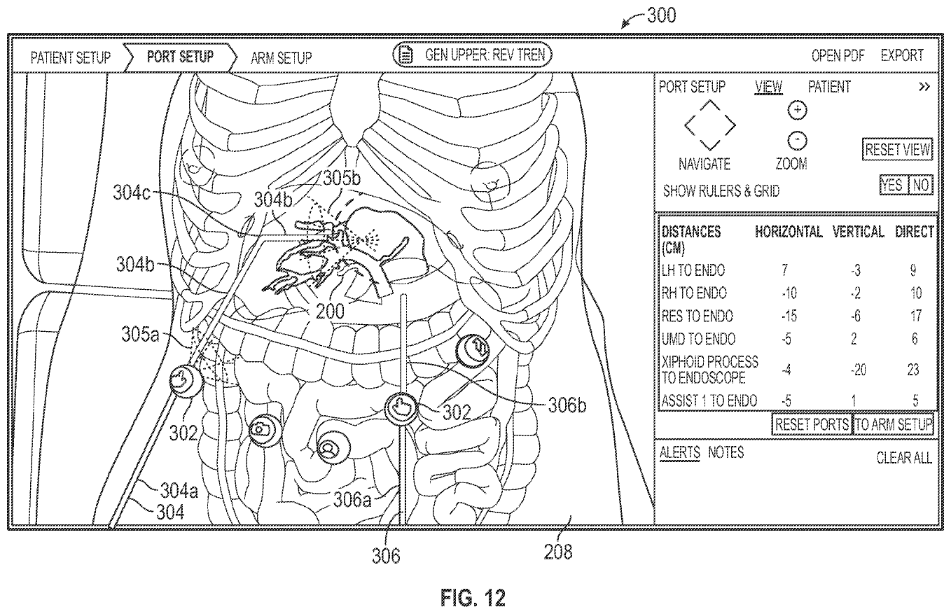

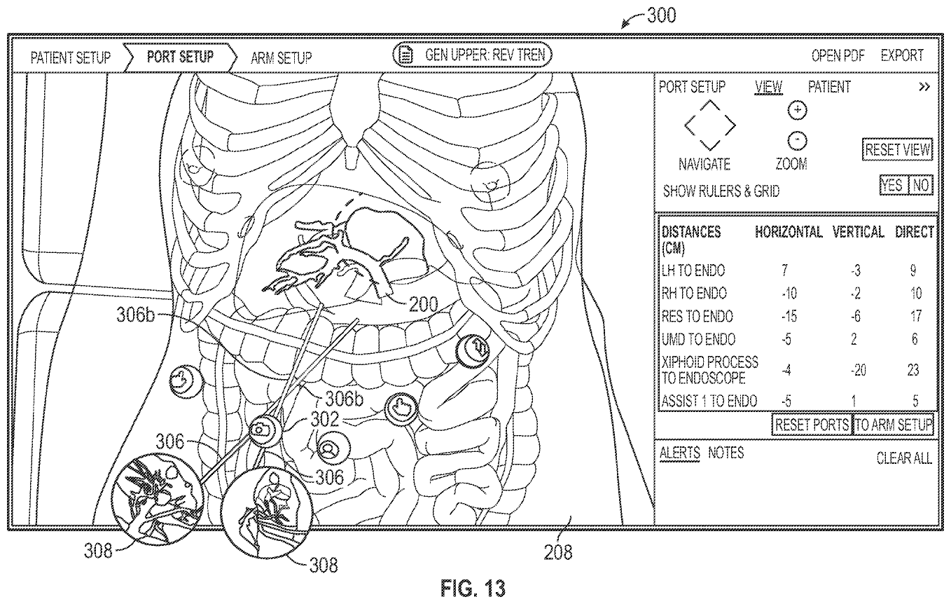

The simulation process is performed at step 114 and is shown in more detail in FIGS. 12 and 13, in which the GUI 300 provides the user with an initial view of the internal 3D model 200. The controller 21a automatically identifies which of the port locations 302 are used by the camera 51 and the instrument 50 and may provide a corresponding icon illustrating the device being inserted into at that port location 302, e.g., camera icon. The GUI 300 provides the user an option to select the icon corresponding to the port location 302 and insert a virtual instrument 50 or a camera 51, which may be shown as insertion models 304 and 306 for the instrument 50 and the camera 51, respectively. The models 304 and 306 includes outside portions 304a and 306a and inside portions 304b and 306b, respectively, which are visually differentiated from each other, e.g., by the degree of transparency, color, etc. to illustrate the portions of the instrument 50 and the camera 51 that are inside the patient.

In embodiments where the insertion model 304 illustrates a device having an articulating joint, e.g., a wristed instrument, the insertion model 304 may also include a joint 304c and an end effector model 304d. To provide additional simulation details, cone 305a and 305b may also be generated by the controller 21a to illustrate the degree of freedom of the insertion model 304 and each joint 304c. The access ports 55a-d limit the movement of the device inserted through about the center of motion, which corresponds to the point of insertion of the access port through the patient. Since the port locations 302 correspond to those points, the cone 305a has its apex at the port location 302 represents the limits of motion of the insertion model 304. Similarly, the cone 305b represents the limits of motion of the end effector model 304d about the joint 304c.

With reference to FIG. 13, the user may place multiple models 306 representing different insertion trajectories of the camera 51 through the single port location 302. Each of the models 306 includes a preview 308 of the endoscopic view of the camera 51. The preview 308 includes a viewpoint of the internal 3D model 200 from the perspective of the model 306. The user may manipulate the models 306, e.g., rotate, advance, retract, etc., and the previews 308 are updated in real time as the models 306 are manipulated. User input may be received through the GUI 300 using a variety of devices, e.g., touchscreen, pointer, keyboards, etc.

The preview 308 shows the modeled organs allowing to the user to confirm whether the proposed optimal camera location is suitable. Variations in patient's anatomy and position deform the organs of the patient. Therefore, the controller 21a is also configured to generate a deformed internal 3D model 200 based on the position and the skeleton model 210 of the patient. The controller 21a may use a neural network trained in pre-operative imaging/modelling of the same organs to learn changes to the shape of organs due to shifting position and orientation of the patient using critical structure landmarks (e.g., vessels, arteries, etc.). The user may select one of the models 306 that was inserted based on a desired view and discard the others. In embodiments, the user may shift the port location 302 to a different location and repeat the preview process to confirm the desired port location 302. The preview window 308 may close during movement of the port location 302 and may automatically reappear once the movement is stopped.

The GUI 300 may also provide playback, i.e., animation, of insertion and internal manipulation of the models 306 to simulate the surgical procedure. The playback may also include movement of the end-effector trajectories to evaluate workspace and collisions with the selected port locations between the camera and the instruments.

With reference to FIG. 6, at step 116, the user confirms the port locations 302, including that of the camera 51, the port locations 302 and the controller 21a generates a setup guide for configuring and positioning the patient on the table 90 and the robotic arms 40 around the patient as shown in FIG. 5 and described above. The guide may include a plurality of text, images, and/or video instructions to be implemented by the operating room staff to setup the surgical robotic system 10. The guide may include instructions for insertion of access ports 55a-d at the port locations 302, instruments 50 to be used, position and orientation of the movable carts 60a-d relative to the table 90, etc.

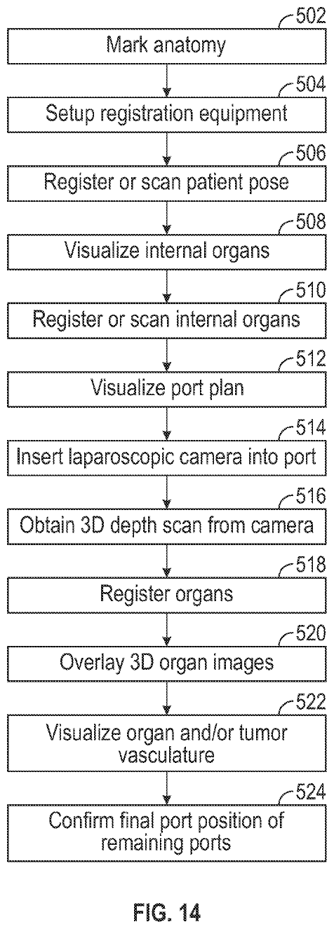

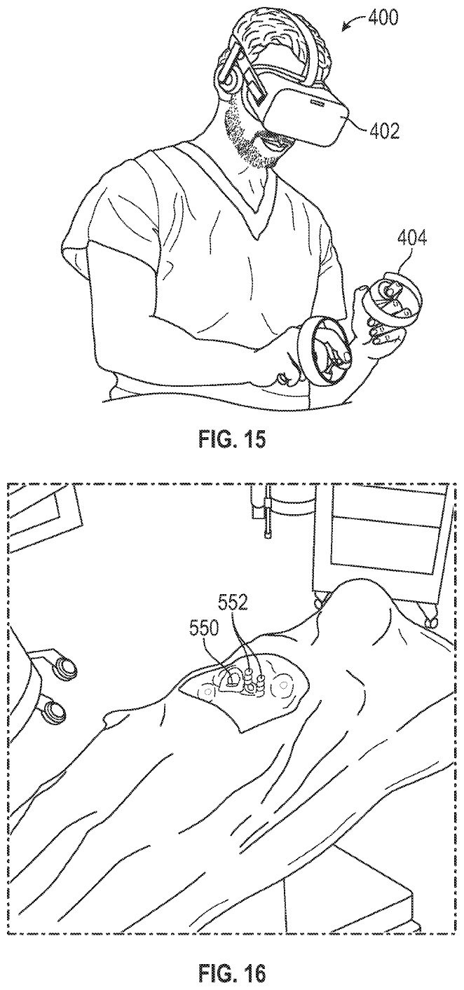

FIG. 14 shows a method of positioning the patient on the table 90 and the robotic arms 40 around the patient as shown in FIG. 5 according to the setup guide generated using the method of FIG. 6. The setup guide generated using the method of FIG. 6 may be implemented using an augmented or virtual reality system 400 of FIG. 15 or displayed on any of the displays 23, 32, 33 of the system 10. In addition, the external vision system 70 is also used, and may include a combination of multiple vision-based sensors 70a-d (RGB-D, LiDAR, or time of flight cameras) mounted on the movable carts 60a-d and/or robotic arms 40a-d as shown in FIG. 5. Data from these vision sensors 70a-d may be processed using individual computers (not shown) having embedded GPU's that are mounted alongside the vision sensors 70a-d. The output from the vision sensors 70a-d is transmitted from each movable cart 60a-d to the tower 20. This alleviates the need to transfer raw data thereby reducing the communication bandwidth bottleneck.

With reference to FIG. 15, the virtual reality system 400 includes a head-mounted display 402 worn by a user around their head and one or more optional handheld controllers 404. The head-mounted display 402 and handheld controllers 404 may generally enable a user to navigate and/or interact with a virtual robotic surgical environment for placing access ports 55a-d and performing other setup steps. The head-mounted display 402 and/or handheld controllers 404 may communicate with the controller 21a via a wired or wireless connection.

In some embodiments, the head-mounted display 402 and/or the handheld controllers 404 may be modified versions of those included in any suitable virtual reality hardware system that is commercially available for applications including virtual and augmented reality environments, such as HOLOLENS® from Microsoft, of Redmond, WA, META QUEST PRO® available from Meta, of Menlo Park, CA. In embodiments where an enclosed virtual reality set is being used, front facing cameras may be disposed on the head-mounted display 402 to provide a front-facing view (e.g., external images of the patient) to be displayed on the head-mounted display 402 along with virtual or augmented projections. In embodiments, where the head-mounted display 402 is an augmented reality display having a projection window, only projections are displayed on the screen.

The head-mounted display 402 and/or the handheld controllers 404 may be modified to enable interaction by a user with a virtual robotic surgical environment (e.g., a handheld controller 404 may be modified to operate the surgeon console 30). In some embodiments, the virtual reality system 400 may further include one or more tracking emitters as part of the external vision system 70 that emit infrared light into the workspace. The tracking emitters may, for example, be mounted on a wall, ceiling, fixture, or other suitable mounting surface. Sensors, markers, or reflectors may be coupled to outward-facing surfaces of the head-mounted display 402 and/or handheld controllers 404 for detecting the emitted infrared light or reflecting the light back to the external vision system 70. Based on the location of any sensors that detect the emitted light and when such sensors detect the emitted light after the light is emitted, the controller 21a may be configured to determine (e.g., through triangulation) the location and orientation of the head-mounted display 402 and/or handheld controllers 404 within the workspace. In other embodiments, other suitable means (e.g., other sensor technologies such as accelerometers or gyroscopes, other sensor arrangements, etc.) may be used to determine location and orientation of the head-mounted display 402 and handheld controllers 404.

In some embodiments, the head-mounted display 402 may include straps (e.g., with buckles, clastic, snaps, etc.) that facilitate mounting of the display 402 to the user's head. For example, the head-mounted display 402 may be structured like goggles, a headband or headset, a cap, etc. The head-mounted display 402 may include two eyepiece assemblies providing a stereoscopic immersive display, though alternatively may include any suitable display.

The handheld controllers 404 may include interactive features that the user may manipulate to interact with the virtual robotic surgical environment. For example, the handheld controllers 404 may include one or more buttons, triggers, touch-sensitive features, scroll wheels, switches, and/or other suitable interactive features. Additionally, the handheld controllers 404 may have any of various form factors, such as a wand, pinchers, generally round shapes (e.g., ball or egg-shaped), etc. In some embodiments, the handheld controller may include a carried device (e.g., wand, remote device, etc.) and/or a garment worn on the user's hand (e.g., gloves, rings, wristbands, etc.) and including sensors and/or configured to cooperate with external sensors to thereby provide tracking of the user's hand(s), individual finger(s), wrist(s), etc. Other suitable controllers may additionally or alternatively be used (e.g., sleeves configured to provide tracking of the user's arm(s)).

The port placement guidance is provided in the form of visual overlay on real-time patient anatomy. The visualization system may be used with various hardware setups, which may be used alone or in combination. In one configuration, any of the displays 23, 32, 34 of the system 10 are used as the visualization device. In another configuration, the head-mounted display 402 is used for visualization. In yet another configuration, the displays are used along with the head-mounted display 402. The video stream from the external vision system 70 is displayed with the guidance overlays on the displays 23, 32, 34 of the system 10 and/or the head-mounted display 402. The combined video output (i.e., stream with the overlays) provides the surgeon with augmented guidance for port placement. The video stream from the head-mounted display 402 is also displayed on the displays 23, 32, 34 of the system 10 simultaneously for the support staff to gain situational awareness as well as for training purposes.

The port placement guidance is provided during initial setup of the system 10 and includes guidance for each of the movable carts 60a-d to be setup at specific distances and angles with respect to the table 90 according to the procedure guidance. The system 10 also provides visual feedback to the setup staff in the form of visual overlay and guidance to move the movable carts 60a-d and their robotic arms 40a-d including the setup arms 61.

The port placement guidance uses the external vision system 70 to automatically detect exposed human anatomy. The controller 21a then segments the surface of human anatomy, estimates the human pose, and predicts an internal skeleton location as described above. The patient-specific pre-op CT model is then registered and visualized on the external human anatomy. Finally, a visual overlay is displayed on the displays 23, 32, 34 of the system 10 and/or the head-mounted display 402 to the surgeon to make incisions for port placement. This guidance considers the number of robotic arms 40a-d, their placements, angles, and the probability of collisions between arms during surgical procedure based on the patient-specific pre-op model. After the laparoscopic access port 55a has been placed, during the “initial look” phase, the sub-tissue tumor and critical structures are also overlayed on the images from the camera 51 to help surgeons visualize internal anatomy and change the port plan if needed.

With reference to FIG. 14, after the patient is brought into operating room and transferred and positioned onto the table 90 at step 502 patient's anatomy is marked by placing fiducial markers on the patient at desired locations. The fiducial markers are used by the external vision system 70 to detect the patient and determine the pose. In lieu of fiducial markers, the external vision system 70 may identify specific features (e.g., joints) of the patient.

At step 504 the registration equipment, e.g., the external vision system 70, is configured to confirm all its components are communicating with each other and are aligned for registering the patient and the system 10. At step 506, the external vision system 70 is used to scan and/or register the patient as well as estimate or predict the patient's pose. Patient's pose may be determined using machine learning, which may include using CNN and/or a ViT backbone to extract features of the pose along with a lightweight decoder for pose estimation. The CNN or ViT may be trained on previous data, for example, synthetic and/or real images of patients in various poses. The pose may be used to generate an external 3D model 208 of the patient as shown in FIG. 10. The external 3D model 208 includes a virtual skeleton model 210 with a plurality of keypoints 212 corresponding to the joints of the patient's anatomy. The external 3D model 208, the skeleton model 210, and the keypoints 212, which may also be generated using various machine learning algorithms trained on datasets including synthetic and/or real images of patients in various poses.

At step 508, internal organs, such as liver, are visualized by projecting a 3D image of the liver 550 on the displays 23, 32, 34 of the system 10 and/or the head-mounted display 402 as shown in FIG. 16. At step 510, the abdomen and the pose of the patient are registered and/or scanned again. At step 512, the access port plan of the setup guide is also visualized by projecting locations 552 corresponding to the port locations 302 of FIGS. 11 and 12.

At step 514, the patient is insufflated and the first access port 50a for the camera 51 is inserted at the projected location followed by the camera 51 being inserted through the access port 50a. The camera 51 is then used in an ‘initial look’ phase at step 516, where the camera 51 is images the surgical site and the video feed is used to generate a dense depth map of the surgical site. The camera 51 may be a stereo endoscope and the controller 21a is configured to generate the depth map using a machine learning algorithm based on the stereoscopic video feed. The controller 21a may track the endoscope location via robotic arm kinematics and Visual-Simultaneous Localization and Mapping (Visual-SLAM) to generate the real-time 3D poses of the endoscope. Furthermore, the controller 21a may generate an extended stitched intra-operative 3D depth map of the surgical site from a plurality of laparoscopic camera poses using a combination of robot arm kinematics, Visual-SLAM, point cloud generation, and sequential point cloud registration.

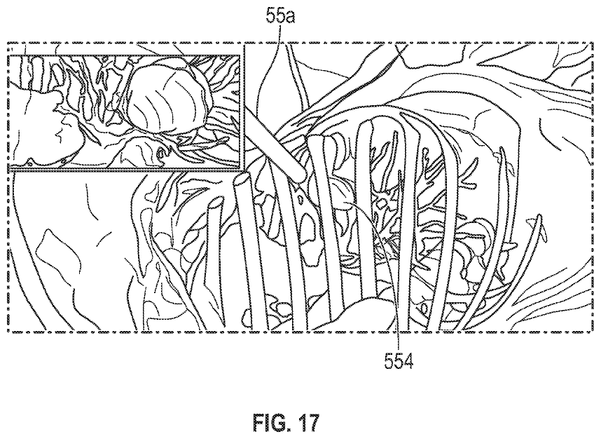

At step 518, patient's organs and surgical site are registered and/or scanned using the camera 51. The controller 21a may initialize the registration of the pre-operative internal 3D model of the patient with the depth map either via point-to-point semi-automatic registration or via fully automatic registration. For semi-automatic registration the user may mark a plurality of points on the pre-operative 3D model and the corresponding points on the 3D surface generated from the camera 51. The controller 21a may use a machine learning model in the form of a neural network to register the pre-operative 3D model and the 3D surface generated from the depth map of stereo images from the camera 51. Such a neural network may be trained to account for the movement of organs resulting from the insufflation of the abdomen. The controller 21a may additionally update the registration between the pre-operative internal 3D model of the patient with the intra-operative 3D depth map via fully automatic registration using real-time robotic arm kinematics data and Visual-SLAM. At step 520, the patient's internal 3D model 200 is overlaid by the controller 21a over the registered patient's organs. At step 522, the internal organs are visualized by projecting a 3D image of the organs 554 on the displays 23, 32, 34 of the system 10 and/or the head-mounted display 402 as shown in FIG. 17.

At step 524, the final port locations 552 are confirmed and the remaining one or more access ports 55b-d may be inserted through the marked port locations 552 under direct vision via the camera 51 to avoid the trocars piercing through any of the internal anatomical organs. The process of generating real-time sequentially registered intra-operative textured 3D model of surgical site during initial look phase creates a non-occupancy volume showing the distance between the abdominal cavity wall and internal organs. The controller 21a may predict the trajectories of trocars inserted through the additional ports and the GUI may display an augmented reality overlay of trocars inserted through the port locations. The GUI may further display warnings and alarms if the projected trajectory or end points of any of the inserted trocars through any of the additional ports may collide with the internal organs as shown in the non-occupancy volume. The GUI may also let the clinical staff move the virtual viewpoint of the stereo endoscope camera using the 3D surgical site model to take a closer look at the projected trocar trajectories through any of the additional ports.

After the final port locations 552 are confirmed guidance for each of the movable carts 60a-d is also provided so that the carts 60a-d are set up at specific distances and angles with respect to the table 90 as shown in FIG. 5. The system 10 also provides visual feedback to the setup staff in the form of visual overlay and guidance to move the movable carts 60a-d and their robotic arms 40a-d including the setup arms 61.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm holding a laparoscopic camera inserted through an access port;
a controller configured to:
generate a port location for an access port on a 3D model of a patient; and
generate a patient-specific setup guide for configuring the access port and the robotic arm;
an external camera configured to register the robotic arm and the patient; and
a display configured to output the port location of the access port as an overlay over an external image of the patient based on registration of the robotic arm and the patient.

2. The surgical robotic system according to claim 1, wherein the display is at least one of a monitor or a head-mounted display.

3. The surgical robotic system according to claim 1, wherein the external camera is further configured to capture a plurality of external images of a patient and images of the robotic arm.

4. The surgical robotic system according to claim 3, wherein the laparoscopic camera is configured to capture internal images of a surgical site.

5. The surgical robotic system according to claim 4, wherein the controller is further configured to generate a depth map of the surgical site from the internal images of the surgical site

6. The surgical robotic system according to claim 5, wherein the controller is further configured to generate a registration of preoperative imaging data of the patient with the depth map.

7. The surgical robotic system according to claim 6, wherein the controller is further configured to track location of the laparoscopic camera via kinematics of the robotic arm and a visual-simultaneous localization and mapping (visual-SLAM).

8. The surgical robotic system according to claim 7, wherein the controller is further configured to update the registration between the preoperative imaging data with the depth map via fully automatic registration using real-time robotic arm kinematics data and the visual-SLAM.

9. A method for assisted access port placement, the method comprising:
capturing images at a laparoscopic camera held by a robotic arm and inserted through an access port;
generating, at a controller, a port location for an access port on a 3D model of a patient;
generating, at the controller, a patient-specific setup guide for configuring the access port and the robotic arm;
generating, at the controller, a registration of the robotic arm and the patient based on external images received from an external camera; and
outputting on a display the port location of the access port as an overlay over an external image of the patient based on registration of the robotic arm and the patient.

10. The method according to claim 9, wherein the port location of the access port is output as the overlay on at least one of a monitor or a head-mounted display.

11. The method according to claim 9, further comprising capturing a plurality of external images of a patient and images of the robotic arm through the external camera.

12. The method according to claim 11, further comprising capturing internal images of a surgical site through a laparoscopic camera inserted through the access port.

13. The method according to claim 12, further comprising generating, at the controller, a depth map of the surgical site from the internal images of the surgical site.

14. The method according to claim 13, further comprising generating, at the controller, a registration of preoperative imaging data of the patient with the depth map.

15. The method according to claim 14, further comprising tracking location of the laparoscopic camera via kinematics of the robotic arm and a visual-simultaneous localization and mapping (visual-SLAM).

16. The method according to claim 15, further comprising tracking the registration between the preoperative imaging data with the depth map via fully automatic registration using real-time robotic arm kinematics data and the visual-SLAM.

17. A method for determining access port placement, the method comprising:
receiving a plurality of external images of a patient captured by an external vision system;
generating an external 3D model of a patient based on the plurality of external images;
generating an internal 3D model of the patient based on preoperative imaging data;
generating a combined 3D model based on the external 3D model and the internal 3D model;
determining a port location for at least one access port based on the combined 3D model;
generating a setup guide for configuring at least one access port and a robotic arm;
outputting on a display the port location of the at least one access port and the setup guide as an overlay over an external image of the patient based on registration of the robotic arm and the patient.

18. The method according to claim 17, further comprising generating the external 3D model of the patient based on a depth map of the patient.

19. The method according to claim 17, further comprising generating a skeleton model including a plurality of keypoints for the patient based on the plurality of external images.

20. The method according to claim 19, further comprising generating the external 3D model based on the skeleton model.
